(19) United States
(12) Patent Application Publication
(10) Pub. No.: US 2012/0231089 A1
(43) Pub. Date: Sep. 13, 2012

(54) REVERSING AUTONOMIC NERVOUS SYSTEM DYSFUNCTION BY POTENTIATING METHYLATION

(76) Inventor: Alan Robert Vinitsky, Potomac, MD (US)

(21) Appl. No.: 13/456,288

(22) Filed: Apr. 26, 2012

Related U.S. Application Data
(63) Continuation-in-part of application No. 11/465,597, filed on Aug. 18, 2006.

(51) Int. Cl. A61K 31/714; A61P 25/00; A61K 33/30

(52) U.S. Cl. 424/643; 514/52; 424/722

(57) ABSTRACT

A method for reversing stress-induced dysfunctions of a human autonomic nervous system in accordance with Methylation Priority Principle includes repeatedly administering a methylation-promoting composition composed to promote a hierarchy of methylation functions of the Methylation Priority Principle including uninterrupted methylation cycle recycling of homocysteine to methionine, and uninterrupted processing and removal of metabolic products of stress.

### CROSS REFERENCE TO RELATED APPLICATIONS

This application is a Continuation in Parts based upon, and claims benefits of, copending and co-owned parent U.S. patent application Ser. No. 11/465,597 entitled “REVERSING AUTONOMIC NERVOUS SYSTEM DYSFUNCTION BY POTENTIATING METHYLATION” filed with the U.S. Patent and Trademark Office on Aug. 18, 2006 by the inventor herein. The copendng and co-owned parent U.S. patent application Ser. No. 11/465,597 is incorporated herein by reference.

### FIELD OF THE INVENTION

The present invention pertains to methods and compositions for reversing dysfunction of the human autonomic nervous system by administration of methylation-promoting compositions. In particular, the present invention describes a method based on administration of compositions that promote uninterrupted recycling of homocysteine to methionine and uninterrupted processing and removal of metabolic products of stress.

### BACKGROUND OF THE INVENTION

The autonomic nervous system (ANS), including branches of the sympathetic nervous system (SNS) and the parasympathetic nervous system (PSNS), terminates the stress response and regulates bodily functions. Generally, the stress response involves activation, of the SNS and requires energy expenditure, whereas termination of the stress response often, occurs via the PSNS, which is calming,, healing and energy-conserving. PSNS is also responsible for regulating most bodily functions [Vinitsky A R, Healthy Aging, 1(5), pp. 63-66 (2006)].

The ability to methylate via the methionine cycle is available in every cell [Finklestein J D, Eur J Pediatr, 157 (Suppl 2), pp. S40-S44 (1998)]. Methylation processes can be ranked (identified as the Methylation Priority Principle—MPP as given in Table 1.) according to the body's immediate needs for survival—alert for stress, rid the body of stressor(s), reverse the alert response when the stress is cleared, maintenance of bodily functions, repair and healing, and last, energy storage [Vinitsky A R, Healthy Aging, 1(6), pp. 75-78, (2006)]. When the resources for methylation are limited, those functions that are not immediately necessary for survival or stress reversal will be reduced or suspended. The consequences of this principle account for the evolution of regional and generalized autonomic dysfunction. Acute and chronic medical conditions and symptoms then develop, resulting from inability to effectively regulate autonomic functions, failure to repair and heal, grow and store energy. In other words, less important methylations can and will be deferred until stress is resolved.

Ideally, the body's response to stress should be immediate, brief (but in no instance longer than necessary) and with the appropriate magnitude. Likewise, termination of the stress response should occur at the right time and with the appropriate magnitude. The end result of these interactions should be a return to the previous state of health. By definition, optimal health is that ideal state of ANS responsiveness to regulate stress responses.

Less than ideal immediate stress responses result in adaptive changes and metabolic consequences. These may include lipid peroxidation [Thiele G, et al,, Comparative Hepatology 3(Suppl 1), p. S25, (2004)], increased oxidised glutathione [James S J, et al., Am J Clin Nutr 80, pp. 1611-17, (2004)] and the production of nitric oxide via the inducible nitric oxide synthase pathway [Pall M., Environmental Health Perspectives, 111(12), pp. 1461-4, (2003)]. Sustained stress results in excess production of stress metabolites and depletion of necessary protective molecules [Brouwer M, et al., Blood 88(5), pp. 1857-64, (1996)]. For example, during the inflammatory phase of their respiratory distress, asthmatics exhale increased levels of aldehydes (Adh) and decreased levels of glutathione (GSH). Following treatment with prednisone, Adh declines and GSH rises [Corradi M, et al., Am. J Respir Crit Care Med 167, pp. 395-99, (2003)].

Methylation (Table 1) is required in the body's generalized response to stress and its termination of the response. For example, heightened and sustained stress alerting requires adrenal responses to produce additional adrenalin and excess SNS activity. Adrenalin is activated by methylation. In turn, adrenalin must be inactivated by methylation. Increased PSNS activity to counterbalance SNS activity requires additional production of acetylcholine (Ach), which requires methylation for its production.

TABLE 1
Methylation Priority Principle—MPP.

| FUNCTION | METHYLATION FUNCTION |
|---|---|
| Alert for Stress | Activate ADRENALIN, increase |
| | sympathetic activity |
| Reverse the Alert for Stress | Inactivate ADRENALIN. Increase |
| | parasympathetic activity; make |
| | choline for ACETYLCHOLINE |
| Remove the Stress or the response | |
| to Stress | |
| Allergy - Histamine release | Inactivate HISTAMINE |
| Metals—mercury, arsenic, tin, | Metabolize for excretion |
| selenium & others | |
| Toxins | Metabolize for excretion |
| Niacin | Inactivate |
| Estrogen | Metabolize |
| Maintenance of bodily functions | Inactivate DOPAMINE, |
| (examples): | NOREPINEPHRINE and |
| Blood pressure, pulse | SEROTONIN, |
| Breathing | depending on needs of |
| Bowel habits | autonomic function |
| Urine habits | Activate MELATONIN; inactivate |
| Mood, memory, concentration, | NOREPINEPHRINE more and |
| balance, movement | SEROTONIN less. |
| Sleep | |
| Repair and Heal | CREATE RNA for protein |
| | synthesis. Proteins for |
| | growth |
| | remodeling (skin, bone, gut |
| | lining) |
| | enzymes for bodily functions |
| | REPAIR DNA |
| Energy Storage | Form CREATINE |

The Methylation Priority Principle (MPP) indicates that methylation resources of the body are allocated by higher priority toward stress responses at the expense of routine bodily functions, whenever there are inadequate resources to sustain methylation for all functions. Consequently, there will be gradual or abrupt onset of symptoms of body malfunction that may be transient, intermittent, prolonged or progressive.

The following symptoms are features of ANS dysfunction in its earliest stages of evolution: (1) localised or regional dysfunction—pain, temperature or vascular dysregulation; organ dysmotility, such as gastrointestinal and genitourinary; cardiovascular dysfunction—blood pressure alterations and pulse irregularities; neuro-motor, sensory, cognitive or psychosocial dysfunctions; hematologic, immunologic, dermatologic or secretory dysfunctions; (2) generalized dysfunction—multiple, regional dysfunctions occurring simultaneously, in stages, sequences or complex patterns.

Examples of regional dysfunction include so-called vasomotor rhinitis, palpitations, tics, constipation or urinary urgency. Generalized dysfunctions may include such end-stage autonomic dysfunction as syncope, and postural tachycardia. Well-recognized medical conditions [Toth C, and Zochodne D W., Seminars in Neurology 23(4), pp. 373-380, (2003)] often have autonomic neuropathy as a manifestation, such as diabetes, multiple sclerosis, and Parkinson syndrome.

Prolonged decompensated stress—intermittent or continuous—is frequently unrecognized. Those individuals with undetected stress may suddenly experience a serious morbidity, a life-threatening or life-ending event. They have undisclosed ANS dysfunction, just as those who present with ongoing symptoms.

The consequence of prolonged stress is failed or inadequate methylation, resulting in ANS dysfunction, and ultimately failure to repair, heal and store energy. Failure to repair and heal results in failed protein synthesis, furthering bodily malfunction, such as enzyme synthesis for complete digestion. Incomplete digestion results in reduced availability of essential amino acids, essential fats and carbohydrates, thereby exacerbating ANS decline.

Status of ANS activities can be directly measured. One computerized diagnostic technique was developed by “The Ansar Group, Inc.”, of 240 South 8^{th} Street, Philadelphia, Pa. The ANSAR® technique simultaneously assesses PSNS and SNS functions by digitally monitoring and recording respiratory and heart rates during rest and challenges specially designed to affect SNS and PSNS branches in contrasting ways. By performing real time digital bi-spectral analysis of recorded signals the ANSAR® program can directly measure spectral powers in different frequency domains of heart rate signals and use it for quantitative evaluation of activities of the SNS and PSNS branches of ANS.

In contrast to the prior art (for example U.S. Patent Application Pub. No. 20050171034 entitled “COMPOSITIONS AND METHODS FOR THE REGULATION OF HOMOCYSTEINE LEVELS WITHIN THE BODY”, by Halevie-Goldman), the current invention includes optimizations of the efficiency of the recycling loop to infinitely generate S-Adenosylmethionine (SAMe), to satisfy the more than 50 Methylation pathways and to provide for the generation of Polyamines, which promote nerve healing.

The current invention incorporates several embodiments pertinent to treatments of patients having normal or low Homocysteine levels. Therefore, according to teachings of the aforementioned Patent Application Pub. No. 20050171034 centered on treatment of elevated Homocysteine levels, no relevant treatment would be indicated. This may be misguided or negligent, as many patients with insufficient methylation may already begin with low Homocysteine. In addition, the Patent Application Pub. No. 20050171034 teaches treatment of high Homocysteine level, but does not address cases pertinent to patients having normal or suppressed Homocysteine, while B12 and folate levels may be already elevated.

Furthermore, the Patent Application Pub. No. 20050171034 teaches that the recited treatment will treat other medical conditions, but offers no way to determine who needs what doses and when to initiate treatment, does not address stress marker cleanup, suggest or imply needs to provide scavengers of stress metabolic products. Instead, the Patent Application Pub. No. 20050171034 introduces other supplements and herbs to compensate for his overmethylated state, in Bipolar and Schizo-affective disorder treated with success using the methods and active ingredients of the current invention.

### SUMMARY OF THE INVENTION

A method for reversing stress-induced dysfunctions of a human autonomic nervous system in accordance with Methylation Priority Principle includes repeatedly administering a methylation-promoting composition composed to promote a hierarchy of methylation functions of the Methylation Priority Principle including uninterrupted methylation cycle recycling of homocysteine to methionine, and uninterrupted processing and removal of metabolic products of stress. The methylation-promoting composition incorporates a first compound chosen from, a group of compounds consisting of folic acid, folates, folinic acid, folinates, dihydrofolate, methyltetrahydrofolate and their mixtures and combinations; and a second compound chosen from a group of compounds consisting of hydroxocobalamin, aquacobalamin, glutathionylcobalamin, adenosylcobalamin and their mixtures and combinations. The first compound and the second compound are arranged for a substantially simultaneous administration and administered transbuccally using multiple unit doses to directly enter a human body circulation for substantially simultaneous elevations of a tissue concentration of the first compound and a tissue concentration of the second compound. The methylation-promoting composition is composed as being free of preservatives or excipients that affect diuresis, and preservatives, or excipients that directly effect the human autonomic nervous system.

### BRIEF DESCRIPTION OF THE DRAWINGS

FIG. 1 is a simplified schematic diagram of basic biochemical processes on which, the current invention is based.

FIG. 2 is a simplified schematic diagram of biochemical processes pertinent to the current invention.

FIG. 3 is a simplified schematic diagram of biochemical processes pertinent to the current invention.

### DETAILED DESCRIPTION OF THE INVENTION

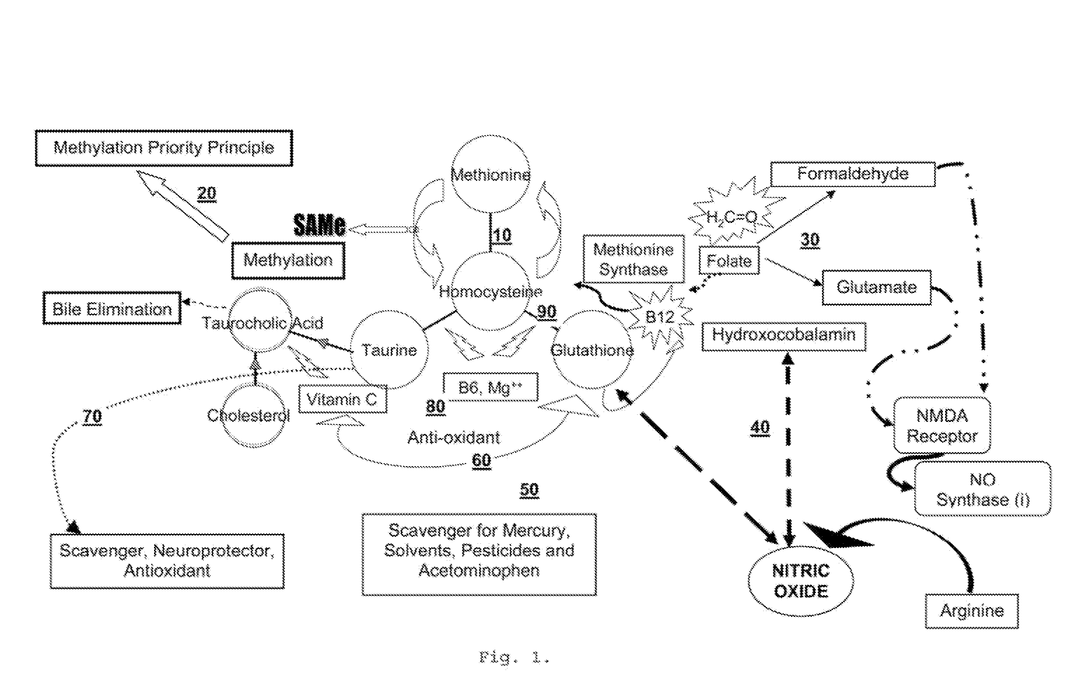

FIG. 1 schematically represents basic biochemical processes on which the current invention is based, The schematic diagram in FIG. 1 is simplified to demonstrate significant relationships between compounds used in compositions and methods for reversing dysfunction of the human autonomic nervous system in the current invention. Consequently, the schematic in FIG. 1 is not a representation of the mechanism of action of disclosed compounds and methods.

The methylation cycle 10, Methionine→S-Adenosylmethionine→S-Adenosylhomocysteine (denoted by standard abbreviation SAMe)→Homocysteine→(remethylation involving Hydroxocobalamin and folate) back to Methionine in FIG. 1 is a part of normal, cell function well known in prior art. For example, U.S. Pat. No. 6,583,123 to Henderson et al. discloses use of S-Adenosylmethionine (SAMe) for treatment and for reducing inflammation of connective tissue,

During a reaction to stress situations, demand for methyl group donors (like SAMe) suddenly increases, in accordance with the Methylation Priority Principle 20, driven by the need to perform additional “Methylation Functions” (Table 1.) Specific stressors requiring methylation further exacerbate the stressed state, thereby destabilizing the autonomic nervous system.

Metabolically active folates like 5-methyltetrahydrofolate can he created in the body via a complex process known as “liver metabolism” from dietary folic acid (vitamin B9)—complex mixtures of polyglutamate (multiple glutamate molecules attached) conjugate compounds. For the purpose of this invention, dietary supplements denoted as first compound, in less complex forms of folic acid, folates, folinic acid and its salts—folinates (like calcium folinate known as leucovorin calcium) that do not need liver metabolism for transformation to methyltetrahydrofolate are preferred. However, the dihydrofolate form functions exceptionally efficiently as a scavenger for aldehydes and glutamate, hence should be considered, even if it needs to be administered as an active component.

Similarly, supplement denoted as second compound—aquacobalamin, methylcobalamin, glutathionylcobalamin and adenosylcobalamin—biochemically close to the metabolically active Hydroxocobalamin (OH—B12) are preferred for the current invention.

Folate scavenges 30 for aldehydes and glutamate and recycles aldehydes through Hydroxocobalamin to homocysteine. Hydroxocobalamin is reduced by glutathione but also scavenges for nitric oxide (NO) 40 and perosynitrite. Hydroxocobalamin can be inactivated, by NO. Aldehydes, NO and peroxynitrite, if allowed to accumulate, can severely disturb balance and functions of both branches of the ANS.

Glutathione (GSH) scavenges 50 for mercury, pesticides and solvents and metabolizes acetaminophen. Glutathione can be inactivated by NO, can reduce ascorfoate (vitamin C) or can be reduced 60 by it. Taurine scavenges 70 for malondialdehyde, hypochlorite and other chlorine-containing compounds. Taurine, magnesium and pyridoxine (vitamin B6) encourage the production of glutathione when homocysteine is converted 90 to cysteine.

Oxidative stress leads to increased aldehydes, nitric oxide and oxidized glutathione (or less available reduced GSH). Nitric oxide causes increased pain and inflammation, through the inducible nitric oxide synthase pathway, which is potentiated by formaldehyde, glutamate and solvents.

When nutrients are diverted for scavenging or are inactivated alone or in combination, methylation will become inadequate under stress. If Hydroxocobalamin is insufficient or dysfunctional, then methylation cannot proceed. When GSH is insufficient or dysfunctional, Hydroxocobalamin is dysfunctional. In either instance, folate becomes functionally insufficient, even when levels are elevated, because it is saturated with methyl groups and aldehydes. This condition is called the folate trap.

The schematic in FIG. 1, infers that key methylation-promoting substances, reduced Hydroxocobalamin and methyltetrahydrofolate must be simultaneously available in proper ratio to alleviate stress-induced imbalances of ANS caused by increased demands for methylation. In order to efficiently relieve symptoms of ANS imbalance, it is desirable to administer methylation-promoting compounds as proper mixes of respective metabolically active forms, i.e. Hydroxocobalamin and methyltetrahydrofolate, or in forms closely related to respective metabolically active forms like folates, folinic acid, folinates; aquacobalamin, methylcobalamin, glutathionylcobalamin, adenosylcobalamin and their mixtures and combinations.

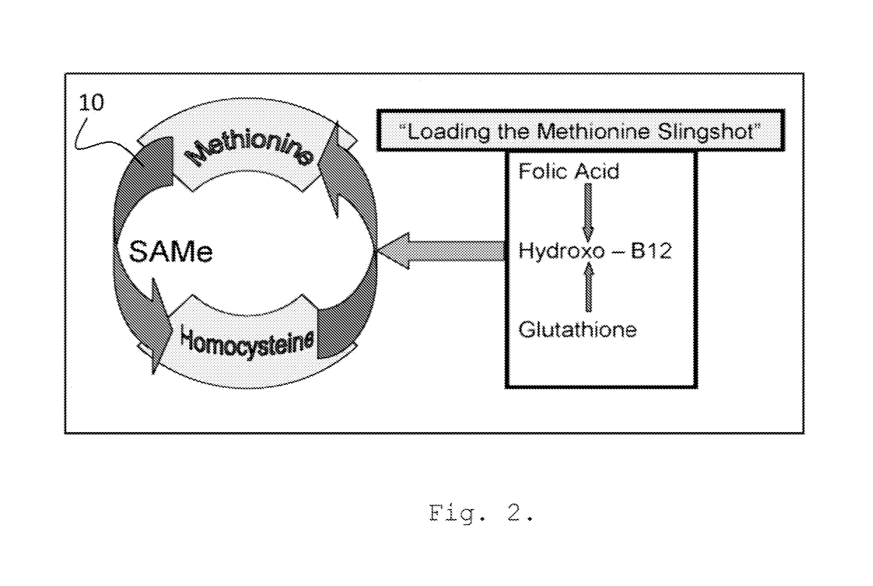

FIG. 2. illustrates in more focused way conversion 90 of Homocysteine to cysteine indicated here by the title-name of “Loading the Methionine Slingshot”. Methionine must ultimately be available to generate SAMe. In this (simplified) representation of the above “Slingshot,” Hydroxocobalamin is activated by reduced Glutathione. Activated Folic Acid may then donate a Methyl group to Hydroxocobalamin yielding MethylCobalamin. These processes under condition of simultaneous abundant supply of metabolically active Hydroxocobalamin, reduced Glutathione, and activated Folic Acid may achieve: Goal #1: MAKE ONE LOOP OF THE METHYLATION CYCLE 10 WORK INFINITELY WELL and Goal #2: MAKE AN INFINITE NUMBER OF LOOPS WORK INFINITELY WELL; in accordance with the MPP of Table 1.

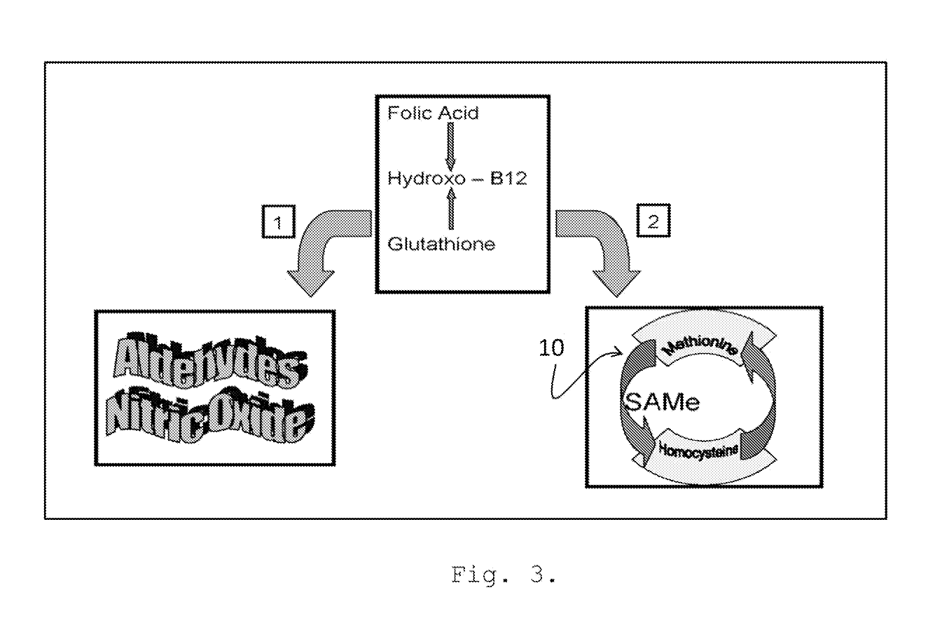

Similarly, FIG. 3 Illustrates in more details stress marker cleanup as a prerequisite to production of SAMe. 1. Folic Acid (Folate) scavenges for Malondialdehyde and other aldehydes. Hydroxcobalamin (OH—B12) scavenges for Nitric Oxide, and Reduced Glutathione is depleted as it scavenges for toxins, such as mercury, pesticides, and solvents, or is oxidized while functioning as an antioxidant. 2. If one or more of these 3 scavengers is in short supply, Methylation may be compromised for a lack of sufficient SAMe. Pharmacologic dosing of Folate and OH—B12 in sufficient doses may be required to clean up aldehydes and nitric oxide. Dose depends on need in accordance with the MPP.

Table 2. provides a schematic summary of the Major Stress Markers which negatively influence methylation by depleting necessary nutrients that promote SAMe production. Sufficient folate, hydroxocoobalamin, and supporting nutrients that redirect homocysteine to supply cysteine for synthesizing glutathione are initially required to correct the stress marker aberrations.

TABLE 2
Major Stress Markers

| | | Clean-up | |
|---|---|---|---|
| Major STRESS MARKERS | | RESPONSE | Treatment |
| 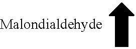 | Oxidative Stress | Folic Acid Scavenger | Folate |
| 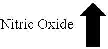 | Inflam- mation Stress | Hydro- xocobalamin Scavenger | Hydro- xocobalamin |
| 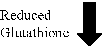 | Scavenges Toxins | Synthesis Re-Reduces | Magnesium, Taurine, Vitamin B6 Ascorbate |

Table 3. pertains more specifically to Mercury removal processes. Mercury adversely affects Methylation by at least 6 mechanisms. Mercury is frequently overlooked as a major source of toxicity, from such sources as dental amalgams. A non-responding patient should he evaluated for mercury “burden”. Mercury is most often stored in fat-soluble tissues, making true toxicity obscured from easy assessment. Inquire if amalgams were previously removed, and if so, when, and what precautions were taken. Flares of symptoms often accompany such “benign” intervention.

TABLE 3
Mercury Disruption
MERCURY DISRUPTIONS in SAMe PRODUCTION and Methylation

Competitively inhibits ATP production
Competitively inhibits COMT utilization of SAMe
Competitively inhibits Methionine Synthase, which prevents it from a
donating Methyl group from Folate through activated Hydroxocobalamin
Activated Hydroxocobalamin
Depletes Glutathione as it scavenges for Mercury
Interrupts synthesis of Glutathione by disrupting Cysteine production from
Homocysteine
Inhibits Paroxonase which recycles Homocysteine Thiolactone to
Homocysteine

Analogous to the Methylation Priority Principle, Arginine (including 1-arginine)or its proxies—Nitric Oxide and Ornithine—exhibits a set of priorities, which rank according to Alerting, Cleansing, Communicating, and Healing functions. Such functions are hierarchically organized in Arginine Priority Principle as shown in Table 4. The Arginine Priority Principle is related to the Methylation Priority Principle as also shown in Table 4. If stress is present, Arginine converts to nitric oxide via Inducible NO Synthase (iNOS). In cleansing mode, in the Krebs Urea Cycle, Urea is generated, as Arginine converts to Ornithine, to rid the body of excess Nitrogen. Nitric Oxide is also generated via Endothelial NOS (eNOS) to vasodilate and Neuronal NOS (nNOS) to communicate via nerves. In healing mode, there are at least three significant effects. Arginine stimulates the pituitary to generate Growth Hormone. Ornithine forms Putrescine, which creates Spermine, when SAMe donates Methionine and further generates Spermidine, with the addition of a second Methionine from SAMe. These polyamines contribute to nerve healing. Finally, Arginine plus Glycine, with the addition of Methyl from SAMe yields Creatine—the energy storage molecule for brain, heart, and muscle.

In alternative embodiments, functions of the Arginine Priority Principle may be supported by administration of Arginine-related compounds including but not limited to ornithine (1-ornithine) and malate salts of arginin and Ornithine and their mixtures and combinations.

TABLE 4
Methylation and Arginine Priority Principles

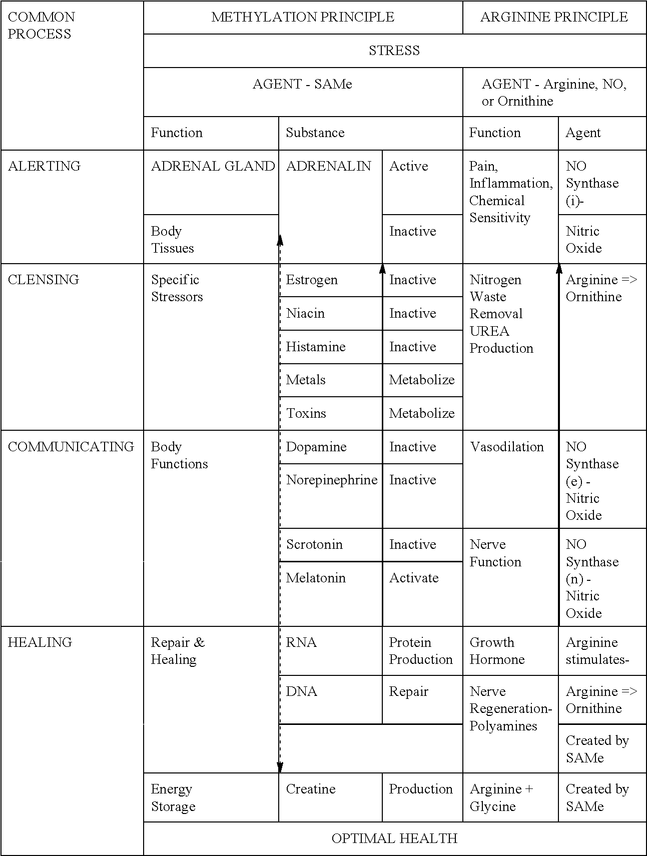

Method of administration may be chosen to circumvent the gastrointestinal tract and first-pass liver metabolism and allows methylation-promoting compounds to directly enter the human body circulation allowing for fast and simultaneous occurrence of high tissue concentrations of ail methylation-promoting components. This requirement makes oral and intramuscular administration methods the least favorable because of long and uncontrollable delays between the time of administration and the time to achieving maximal tissue concentrations. Direct intravenous or subcutaneous administrations, although probably effective, may be inconvenient due to the impracticality of long-term self-administration of multiple doses. However, intermittent intravenous therapy can be a useful therapeutic intervention.

The route of administration of methylation-promoting compositions through mucous membranes (transmucosal) to the proximal circulation pathways satisfies requirements for effective simultaneous delivery. Mucous membranes of the buccal cavity offer convenient and efficient access of transbuccally administered compositions to the body circulation. It is conceivable that inhaled (via nebulizer), intranasal or subcutaneous routes may be options, but folic acid has not been sufficiently optimized and tested for administration via these routes.

Folates (folinic acid, folinic acid and folinates) and cobalamins (Hydroxocobalamin, aquacobalamin, methylcobalamin, glutathionylcobalamin and adenosylcobalamin) are known dietary supplements available commercially in a liquid (solutions), powder, or tablet form. In addition, a gel form for transmucosal administration may be of interest in embodiments directed toward use in long duration sporting events like marathons, “Iron Man” events, etc.

Because of a wide variation in optimized single doses and possibility for progressive improvements in doses and ratios of active ingredients, it is frequently desirable to store folates and cobalamins separately and combine them into a particular methylation-promoting composition just prior to the time of simultaneous administration. It may be also understood that particular combinations may be standardized in stable fixed-dose combinations of interest.

It is evident from Tables 1-4 and FIGS. 1-3, that methylation-promoting compositions and compounds participate in a multitude of frequently competing methylation processes. Because of composition's complicated metabolism, single unit doses and total daily doses can vary significantly, most frequently as a function of a) individual symptoms, b) relief of symptoms, c) wear-off effect, (i.e. return of symptoms), d) adverse reactions or side effects of a treatment dose and e) prophylactic dosing. Listed factors further depend on patients' individual conditions, such as severity and frequency of exposures to stressors, age, general state of fitness, diet, rest, etc.

Single unit dose of said first compound ranges from 0.5 mg to 1000 mg. Single unit dose of said second compound ranges from 0.2 mg to 500 mg. Single unit dose mass ratio of said first compound versus said second compound ranges from 0.5 to 5.

Depending on individual dosing and treatment schedule, the cumulative daily dose is likely to change, possibly daily. During applications of methods in accordance with the present invention total daily dose did not exceed 10000 mg of said first compound and 5000 mg of said second compound. Similarly, total number of administered unit doses per day did not exceed 750 unit doses for adult and 300 unit doses for children 13 years of age or younger.

Patients' monitoring did not indicate contraindications associated with doses consisting of upper limits of listed ranges. Even higher doses are considered applicable to some particularly severe conditions of ANS imbalance at least for short duration as part of a prolonged regimen.

Inspection of schematics in FIG. 1 may indicate that methylation-promoting functions require vitamin C 60, Taurine, and B6-Mg++ combination 80. If those ions and compounds are deficient, the methylation-promoting composition comprising said first compound and said second compound would not be effective, regardless of the dosing and administration method. It is in accordance with the current invention to supplement the deficient additives together with said first and second compound. The methylation-promoting composition in accordance with this invention was successfully combined with multiple vitamin and mineral supplements containing B complex vitamins, fat-soluble vitamins, magnesium, zinc, biotin and their mixtures and combinations.

Performance of “methylation functions” in Table 1. is contingent on the availability of multiple enzymes, cofactors and dietary supplements. If those compounds are not sufficiently available, one needs to supplement them as additions to the methylation-promoting composition comprising said first compound and said second compound. The methylation-promoting composition including the first compound and the second compound can be successfully supplemented by taurine, glutathione reduced, essential amino acids, arginine, ornithine, essential fatty acids, anti-oxidants, calcium, iron, copper, selenium, chromium, vanadium, manganese, molybdenum, boron, iodine/iodide, phosphorus/phosphate, phospholipids, dimethylaminoethanol, inositol, dimethylglycine, betaine, gamma-amino butyric acid, natural hormone replacements, digestive enzymes and probiotics and their mixtures and combinations. Other compounds and dietary supplements can also be added without exceeding the scope of the present invention.

Optimal recycling of Homocysteine turns off protein destruction and encourages protein production. Thiolactone hits on Lysine residues in proteins thereby denaturing them. Evidence of chronic patterns of amino acid depletion, is found in 24 hr urine specimens. The first four may be substantially uniform. An occasional individual may have normal phosphoethanolamine. Children may have increased Asparagine transiently. Increased Amino acids usually suggests a dumping (wasting) pattern. As an example, acute protein breakdown has high Lysine consistent with the Thiolactone destructive pattern.
i. Phosphoethanolamine
ii. Hydroxyproline
iii. Asparagine
iv. Isoleucine
v. Glutamine
vi. Tyrosine
vii. Phenylalanine
viii. Methionine (often low normal)
ix. Serine
x. Glycine
xi. Valine
xii. Arginine

Protein synthesis improves with optimal function, resulting in reduction of autoantibodies (thyroid peroxidase, antithyroglobulin antibody, thyrotropin receptor II antibody, rheumatoid factor, and ANA), improved antibody production to fight infections (e.g. Epstein-Barr, Cytomegalovirus, Toxoplasmosis, and positive Lyme Western Blot). Previously undetectable (masked) hypothyroidism is observed when adrenal function improves. The present invention permits supplementation with essential amino acid products (such as those in the U.S. Pat. No. 5,132,113)

In non-methylation embodiments, the administration of Hydroxocobalamin encourages muscle contraction, when the simultaneous administration of either Glutathione-promoting nutrients or reduced-Glutathione is offered. Also, SAMe may convert Putrescine to Spermine, and Spermine to Spermidine, by donating Methionine. These polyamines may promote nerve function but also may function as modulators on the NMDA receptor (Williams Biology of the NMDA Receptor). Recognition of when to supplement with an absorbable form of Glutathione may depend on individual response to Hydroxocobalamin/Folate. Also, the observation of how quickly the sublingual (transmucosal) combination dissolves, and MCV rises during treatment may be considered. Identified prior art offers no guidance or suggestions on how to address this issue.

Furthermore, introduction of Methionine may depend on a decline of Homocysteine to below 6. An intervention to treat mercury burden/toxicity is critical in optimizing the production of SAMe, as Mercury compromises the production of SAMe and its utilization. The above prior art is silent regarding this contingency. Even further, simultaneous administration and delivery of Folate and Hydroxocobalamin may need to be transbuccal, subcutaneous, or intravenous avoiding liver metabolism or delayed absorption. Oral administration may cause delays in delivery of B12. Folate may not be absorbed if there is alcohol in the gut. If administered orally, Folate may also be picked up by Transcobalamin II—the carrier protein; hence, no opportunity for scavenging for Nitric Oxide.

An intramuscular delivery may result in a low release (relative to the above) of the supplements, which precludes their function as scavengers to clean up markers of stress. Folate may clean up oxidative stress (aldehydes) and Hydroxocobalamin cleans up inflammation stress (Nitric Oxide), Methyltetrahydrofolate may not clean up aldehydes. Similarly, Methylcobalamin and Cyanocobalamin do no clean up nitric oxide.

In some embodiments, Glutathione may be depleted because it is oxidized, scavenging for toxins—like metals, especially mercury, pesticides, solvents, vapors, and acetaminophen—or is increasingly degraded by GGT. Supplementation with Taurine encourages the recycling of Homocysteine to Methionine by redirecting the metabolic pathway in that direction. It may also functions as a scavenger of Malondialdehyde. Similarly, Ascorbate (Vitamin C) must be administered in sufficient doses to retain functional reduced Glutathione (60 mg/kg for children), or dependent on personal need (“Ascorbate Flush”), while Magnesium and Vitamin B6 may be provided to promote the production of Glutathione (e.g. Magnesium ˜100 mg up to 1000 mg twice or 3×/day and B6 ˜500 mg daily in divided doses.)

In different embodiments it may be beneficial or strongly recommended, to use, at least at some stages of treatment, ALL B COMPLEX vitamins including Thiamine, Riboflavin, Niacinamide, and Pantothenic acid. NAC further encourages Homocysteine to recycle to Methionine. Homocysteine is converted to Thiolactone as well, but Thiolactone will only recycle to Homocysteine when the enzyme Paraoxonase is upregulated by Methionine. Thiolactone may destroy proteins, promote autoimmunity, and encourage degradation of Acetylcholine, thereby encouraging impairment of muscle function and Parasympathetic Nervous System activity.

Some studies such as HOPE II and Norvit showed no improvement in cardiovascular outcomes when there was modest reduction in Homocysteine. Remethylation cannot function infinitely well when methylcobalamin and tetrahydrofolate are administered, as adrenalin will be generated, but there will be insufficient SAMe to inactivate adrenalin. The doses for Methylcobalamin, as disclosed and suggested in the Patent Application Pub. No. 20050171034, are potentially toxic and intolerable under a substantial majority of human conditions. Furthermore methylation may not proceed infinitely well until Methionine-Homocysteine-Methionine loops are functioning infinitely well. At that stage, an infinite number of those loops must be generated when Homocysteine levels are below 6. Therefore, Methionine may be added to the treatment regimen. Doses may range from 250 mg to 10000 mg daily.

One set of embodiments of the current invention pertains to administration of a mixture (“an all-in-one product”) that may include:
a. 500 mg tablet
b. 0.4% Hydroxocobalamin 2 mg
c. 1% folic acid 5 mg
d. 24.475% calcium carbonate 122 mg [48 mg Ca per tablet]
e. 39% magnesium phosphate 195 mg [39 mg Mg per tablet]
f. 20.5% xylitol 103 mg
g. 5% sodium croscarmellose 25 mg
h. 9.625% microcrystalline cellulose 48 mg.
   Number of doses consumed daily ranges from 1 [unlikely] to 100, most often 6-10 when healthy, and 20-40 when requiring additional support.

An additional composition of a supporting the above all-in-one product may include:
a. P5P 10 mg
b. Taurine 100 mg
c. Mg 28 mg
d. Ascorbate 25 mg
e. Possible addition of DMG and Betaine.
f. Supporting inactive components for delivery

Consequential effects on the autonomic nervous system and other functions may include:
a. Correction of Autonomic Paradoxes
b. Awareness of dehydration
   i. Itchy
   ii. Sneezy
   iii. Runny
   iv. Drippy
c. Reduction in histamine, which is a messenger for both dehydration and allergy.
d. Treatment of allergic symptoms and asthma—the 3 stress markers are present. These 3 are treated with Folate, Hydroxocobalamin, when administered simultaneously and sublingually or transbuccal, while supporting nutrients C, Magnesium, B6 and Taurine promote glutathione
   i. Increased Malondialdehyde
   ii. Increased Nitric Oxide
   iii. Decreased Glutathione
e. Reduction in PMS and menstrual symptoms
f. Treatment of eczema
g. Improved exercise tolerance and endurance
h. Support for improved sleep
i. Support and treatment of attention deficit disorder
j. Support and treatment of autistic spectrum disorder
k. Support and treatment of bipolar disorder
l. Support and treatment of depression and anxiety
m. Support and treatment of blood pressure, arrhythmias
n. Improvement in fatigue, pain, concentration, memory loss.
o. Improvement in immune function.
p. Promotes repair and healing while reducing stress markers.
q. Addition of small amounts of Lithium provide neuroprotection and improves diabetes and other functions.
r. Reduction in the need for medications, because medications work more effectively when stress is reduced,

Inflammation management may be introduced when methylation has reduced stress sufficiently, in situations where toxins or conditions are still active; infections, toxins—metals, solvents, and mold. This may be achieved, for example, by administration of:
a. Quercetin 500 mg up to 2000 mg 4×/day, or even 2000 mg every 15 min×4, then every hour ×4, then 4×/day
b. Green tea decaf—up to 3 cups/cap [based on epigallocatechin gallate—EGCg—in capsule form, maximum 2 caps 3×/day
c. Bromelain 2000 units up to 4 4×/day
d. Curcumin—up to 4× 650 mg 4×/day
e. Fish Oils 1 gm up to 3 gm/day
f. Vitamin D3 up to 10,000 units daily.

Minerals and Anions may also be critical to nutrition correction. Administration of:
b. Copper (200 mcg/drop up to 2 mg daily) to promote Tyrosine production from Phenylalanine, and with Vitamin C to promote Norepinephrine from Dopamine. Also promotes conversion of T4 to T3. Tends to parallel Estrogen levels
c. Zinc 25-50 mg promotes Niacinamide production, encourages Betaine to donate Methyl group to Homocysteine to form Methionine (separate from B12/folate)
d. Selenium 200 mcg-400 mcg daily
e. Copper, zinc, and selenium encourage T4 conversion to T3
f. Chromium 200 mcg 1 twice daily in select individuals
g. Vanadium 2.5 mg 1 twice daily in select individuals
h. Molybdenum 250 mcg 1 daily in select individuals
i. Calcium Hydroxyapatite 250 mg up to 4 daily
   j. Ferrous Iron—up to 150 mg twice daily, depending on individual need
k. Manganese 10 mg 1 daily in select individuals
l. Lithium Orotate 5 mg 1 daily up to 2 twice daily,
m. Iodine/Iodide 12.5 mg 1 up to 4 daily, depending on individual need;
   may be recommended in some embodiments.

Furthermore, anti-oxidants can be administered when methylation is working correctly. Frequently anti-oxidants become pro-oxidants in the wrong setting. Also, biotoxin binders—Brown Seaweed—up to 8×500 mg in 1 or two doses daily, Adrenal Support may include Rhodiola, Magnolia, Perilla Oil; Also Eleuthero (Siberian Ginseng), and/or Gastrointestinal and Volume Support—Licorice˜400 mg up to 2 twice daily may be considered and included for particular needs in some embodiments. Andrographis may be used to reduce the activity of GGT, re: degradation of glutathione, thereby promoting increased glutathione.

The present invention has been described with reference to the above exemplary embodiments. While specific values, relationships, materials and steps have been set forth for purpose of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

We claim:

1. A method for reversing stress-induced dysfunctions of a human autonomic nervous system in accordance with Methylation Priority Principle comprising:
repeatedly administering a methylation-promoting composition, being composed to promote a hierarchy of methylation functions of the Methylation Priority Principle including uninterrupted methylation cycle recycling of homocysteine to methionine, and uninterrupted processing and removal of metabolic products of stress, the methylation-promoting composition including:
a first compound chosen from a group of compounds consisting of folic acid, folates, folinic acid, folinates, dihydrofolate, methyltetrahydrofolate and their mixtures and combinations; and
a second compound chosen from a group of compounds consisting of hydroxocobalamin, aquacobalamin, glutathionylcobalamin, adenosylcobalamin and their mixtures and combinations;
wherein the first compound and the second compound are arranged for a substantially simultaneous administration and administered transbuccally using multiple unit doses to directly enter a human body circulation for substantially simultaneous elevations of a tissue concentration of the first compound and a tissue concentration of the second compound, and
wherein, the methylation-promoting composition is composed as being free of preservatives or excipients that affect diuresis, and preservatives, or excipients that directly effect the human autonomic nervous system.

2. The method for reversing dysfunctions of the human autonomic nervous system of claim 1, wherein the administration of the methylation-promoting composition is performed using a transbuccal administration method arranged to avoid a gastrointestinal tract and a first-pass of liver metabolism.

3. The method for reversing dysfunctions of the human autonomic nervous system of claim 1, wherein the first compound and the second compound are stored separately and combined into the methylation-promoting composition just prior to a time of a simultaneous administration.

4. The method for reversing dysfunctions of the human autonomic nervous system of claim 1, wherein the first compound and the second compound are combined into a unit-dose in a state of liquid, a state of gel, a state of powder, or in a tablet form.

5. The method for reversing dysfunctions of the human autonomic nervous system of claim 1, wherein, a single unit dose for the first compound ranges from 0.5 mg to 1000 mg.

6. The method for reversing dysfunctions of the human autonomic nervous system of claim 1, wherein a single unit dose for said second compound ranges from 0.2 mg to 500 mg.

7. The method for reversing dysfunctions of the human autonomic nervous system of claim 2, wherein a single unit dose mass ratio of the first compound versus the second compound ranges from 0.5 to 5.

8. The method for reversing dysfunctions of the human autonomic nervous system of claim 1, wherein a total number of administered unit doses per day does not exceed 750 unit doses for adult and 300 unit doses for children 13 years of age or younger.

9. The method for reversing dysfunctions of the human autonomic nervous system of claim 1, wherein the methylation-promoting composition consists of a solution comprising 5 mg of the folic acid applied to a 2 mg tablet of the hydroxocobalamin, and wherein the methylation-promoting composition is arranged to be administered by a transbuccal administration method on arising, midday and at bedtime.

10. The method for reversing dysfunctions of the human autonomic nervous system of claim 1, wherein, in addition to the first compound and the second compound, the methylation-promoting composition comprises compounds chosen from a group consisting of multiple vitamin and mineral supplements containing B complex vitamins, fat-soluble vitamins, magnesium, zinc, biotin, and their mixtures and combinations.

11. A method for reversing stress-induced dysfunctions of a human autonomic nervous system in accordance with Methylation Priority Principle and Arginine Priority Principle comprising:
repeatedly administering a methylation-promoting composition, being composed to promote a hierarchy of methylation functions of the Methylation Priority Principle including uninterrupted methylation cycle recycling of homocysteine to methionine, and uninterrupted processing and removal of metabolic products of stress, the methylation-promoting composition including:
a first compound chosen from a group of compounds consisting of folic acid, folates, folinic acid, folinates, dihydrofolate, methyltetrahydrofolate and their mixtures and combinations;
a second compound chosen from a group of compounds consisting of hydroxocobalamin, aquacobalamin, glutathionylcobalamin, adenosylcobalamin and their mixtures and combinations; and
a third compound chosen from a group of compounds consisting of arginin, 1-arginin, ornithine, 1-ornithine, malate salts of arginin and ornithine, and their mixtures and combinations; and
wherein the first compound and the second compound are arranged for a substantially simultaneous administration and administered transbuccally using multiple unit doses to directly enter a human body circulation for substantially simultaneous elevations of a tissue concentration of the first compound, a tissue concentration of the second compound, and a tissue concentration of the third compound; and
wherein, the methylation-promoting composition is composed as being free of preservatives or excipients that affect diuresis, and preservatives, or excipients that directly effect the human autonomic nervous system.

12. The method for reversing dysfunctions of the human autonomic nervous system of claim 11, wherein, in addition to the first, the second, and the third compound, the methylation-promoting composition comprises compounds chosen from a group consisting of multiple vitamin and mineral supplements containing B complex vitamins, fat-soluble vitamins, magnesium, zinc, biotin, and their mixtures and combinations.

13. The method for reversing dysfunctions of the human autonomic nervous system of claim 11, wherein, in addition to the first, the second, and the third compound, the methylation-promoting composition comprises compounds chosen from a group consisting of P5P, Taurine, Magnesium, Ascorbate, DMG and Betaine.

14. A methylation-promoting composition for reversing stress-induced dysfunctions of a human autonomic nervous system in accordance with Methylation Priority Principle comprising:
the methylation-promoting composition arranged to promote a hierarchy of methylation functions of the Methylation Priority Principle including uninterrupted methylation cycle recycling of homocysteine to methionine, and uninterrupted processing and removal of metabolic products of stress, the methylation-promoting composition including:
a first compound chosen from a group of compounds consisting of folic acid, folates, folinic acid, folinates, dihydrofolate, methyltetrahydrofolate and their mixtures and combinations; and
a second compound chosen from a group of compounds consisting of hydroxocobalamin, aquacobalamin, glutathionylcobalamin, adenosylcobalamin and their mixtures and combinations;
wherein the first compound and the second compound are arranged for a substantially simultaneous administration and administered transbuccally using multiple: unit doses to directly enter a human body circulation for substantially simultaneous elevations of a tissue concentration of the first compound and a tissue concentration of the second compound, and
wherein, the methylation-promoting composition is composed as being free of preservatives or excipients that affect diuresis, and preservatives, or excipients that directly effect the human autonomic nervous system.

15. The methylation-promoting composition for reversing dysfunctions of the human autonomic nervous system of claim 14, have been arranged for a transbuccal administration method and composed to: avoid a gastrointestinal tract, and a first-pass of liver metabolism.

16. The methylation-promoting composition for reversing dysfunctions of the human autonomic nervous system of claim 14, wherein the first compound and the second compound are arranged to be stored separately and combined into the methylation-promoting composition just prior to a time of a simultaneous administration.

17. The methylation-promoting composition for reversing dysfunctions of the human autonomic nervous system of claim 14, wherein the first compound and the second compound are combined into a unit-dose in a state of liquid, a state of gel, a state of powder, or in a tablet form.

18. The methylation-promoting composition for reversing dysfunctions of the human autonomic nervous system of claim 14, wherein, in addition to the first, the second, and the third compound, the methylation-promoting composition comprises compounds chosen from a group consisting of multiple vitamin and mineral supplements containing B complex vitamins, fat-soluble vitamins, magnesium, zinc, biotin, and their mixtures and combinations.

19. The method for reversing dysfunctions of the human autonomic nervous system of claim 14, wherein, in addition to the first, the second, and the third compound, the methylation-promoting composition comprises compounds chosen from a group consisting of P5P, Taurine, Magnesium, Ascorbate, DMG, and Betaine.
